SEQUENCE LISTING (3 sequences)

SEQ ID NO: 1 - DNA, 22 bp, Sus scrofa
gcctagccctaaacccaaatag

SEQ ID NO: 2 - DNA, 22 bp, Sus scrofa
gcaagggttggtaaggtctatc

SEQ ID NO: 3 - skipped

(19) United States
(12) Patent Application Publication
(10) Pub. No.: US 2018/0037959 A1
(43) Pub. Date: Feb. 8, 2018

(54) Process using ZEN hydrolysis probe for detection of porcine contamination and a kit thereof

(71) Applicant: Universiti Brunei Darussalam, Gadong (BN)

(72) Inventors: Hamadah LUBIS, Gadong (BN); Minhaz Uddin AHMED, Gadong (BN)

(21) Appl. No.: 15/672,194

(22) Filed: Aug. 8, 2017

(30) Foreign Application Priority Data
Aug. 8, 2016 (BN) BN/N/2016/0062

(51) Int. Cl. C12Q 1/68; G01N 33/12

(52) U.S. Cl. CPC C12Q 1/6888; C12Q 1/6818; G01N 33/12; C12Q 2600/158

(57) ABSTRACT

A rapid, sensitive and cost-effective process of detection of porcine contamination in a food product sample is provided herein. The present invention further provides a real-time polymerase chain reaction (PCR) based process which uses highly specific oligonucleotides primers and ZEN™ probe, and a kit that contains the primers and/or probe useful for rapid detection of porcine DNA in a food sample. The oligonucleotide primers disclosed in the present invention provide amplification of the target gene porcine (Sus scrofa) mitochondrial 12S ribosomal RNA gene in highly sensitive and specific manner showing no cross amplification.

### RELATED APPLICATION

This application claims the benefit of Brunei Application No. BN/N/2016/0062 filed on Aug. 8, 2016 and entitled “A qPCR System for Sensitive Detection of Porcine DNA”, the content of which is incorporated in its entirety herein by reference.

### FIELD THE INVENTION

The present invention is in the technical field of identifying the porcine contamination from meat based raw and processed food sample.

More particularly, the present invention is in the technical field of detecting the porcine contamination by performing polymerase chain reaction (PCR) assay using specific set of primers and probe.

The present invention further relates to a process and a kit using novel set of oligonucleotide primers and a probe for detecting the porcine contamination from meat based raw and processed food sample for Halal authentication.

### BACKGROUND AND RELATED ART

Porcine adulteration of food is objectionable to a sizeable percentage of the global population owing to health concerns and/or religious faiths. However, unintentional or intentional porcine adulteration is common issue worldwide in the food industry. For instance, cheaper meats were used as a substitute for more expensive meats. Most frequently, pork meat has been used to substitute other meat types in food products.

Two billion Muslims worldwide consume Halal foods and they make the halal industry a trillion US dollars business (Alam & Sayuti, 2011). Halal food production industry is growing very fast worldwide with increasing globalization and mobility of halal consumers. Food manufacturers worldwide start to develop halal brands as every manufacturer wants a piece of the increasing halal market which is causing intentional or unintentional adulterations of foods in the form of presence of substances not stated in the food labels or due to the substitution of acceptable food components with unacceptable ones or by owing to the cross-contamination of food components during co-processing within the same industrial process. Meats are very prone to adulterations and mislabeling as different types are processed at the same facilities (Ballin, 2010). Consequently, concerns arise from the perspectives of health, diet, lifestyles, cultures and religions which necessitate the need of species identification in meat containing foods (Fang & Zhang, 2016). Therefore, the identification of animal species especially pork in food products is becoming an important issue to consumers. The implication of misleading the labeling of food can be much more important concerning the presence of potentially non-Halal food. For this reason, a strong demand prevails for a fast and sensitive method to detect and quantify porcine DNA in foods.

Several methods have been developed to identify the species of origin of fresh meat and meat products. Numerous methods based on DNA analysis have been employed in the food industry to monitor adulterations of food products. Methods established for animal speciation are mostly lipid-, protein- and DNA-based. However, DNA-based methods are particularly more reliable as DNA is more stable under conditions associated with the high temperatures, pressures and chemical treatment used in food processing.

Species identification of animal tissues in meat products is an important issue to protect the consumer from illegal or undesirable adulteration for economic, religious and health reasons. For this purpose, numerous analytical methods have been developed based on protein and DNA analysis. Among the DNA-based methods that are highly developed for species identification are species-specific conventional PCR and real-time PCR. Among the targeted gene fragments developed for pork species specific PCR are those derived from 12S rRNA, ND5, Mitochondrial Displacement Loop (D-Loop) and Nuclear Melanocortin receptor 1 (MCIR).

Despite proper labeling, doubts are inevitable due to the past cases of fraudulent labeling by dishonest manufacturers, for example, detection of porcine DNA in a food product that had been labeled pork-free (Demirhan, Ulca, & Senyuva, 2012), Intensified efforts has been visible over the last ten years to develop analytical techniques forthe detection and quantification of the presence of porcine species in meat products especially due to the zero tolerance among the Muslims towards the presence of porcine ingredients in foods.

Protein- and DNA-based electrophoresis, chromatographic (Toorop, Murch, & Ball, 1997), and immunological (Asensio, Gonzalez, Garcia, & Martin, 2008) methods have been employed in species identification. However, due to the characteristic denaturation of proteins at high temperature-protein-based methods showed limited applicability in the detection of species in cooked, baked or heat-treated food products (Cal, Gu, Scanlan, Ramatlapeng, & Lively, 2012; Nakyinsige, Che Man, & Sazili, 2012). In contrary, DNAs 96 are relatively more stable under heat and pressure treatments (Roy, Rahman & Ahmed 2016; Safavieh et al 2016; Roy et al 2016; Ahmed et al 2010) and polymerase chain reaction (PCR) based DNA amplification offers fast, specific and sensitive meat species detection (Cammá, Domenico, Monaco, 2012; Soares et al., 2013; Fang & Zhang, 2016). Generally, end-point PCR amplifies a species specific region of the DNA with the aid of forward and reverse primers and the amplicons are further analysed by agarose gel electrophoresis. Hence, end-point PCR provides only the absence or presence of the concerned species in the sample. On the other hand, real-time PCR or quantitative PCR(qPCR) is a DNA-based species identification method that not only indicates the presence or absence of the target DNA sequence in the sample faster but also quantitate the initial concentration of the target (Navarro, Serrano-Heras, Castao, & Solera, 2015) with higher sensitivity and specificity without any post-PCR analysis (Fang & Zhang, 2016). A number of studies developed real-time PCR assays for detection of porcine and other species in different types of samples. Porcine-specific primers were used to detect porcine DNA in adulterated meatballs (Ali et al., 2012) and in gelatine (Demirhan et al., 2012). Multiplex real-time PCR assays based on primers specific to porcine, chicken and bovine species were developed to identify chicken and turkey in meat mixtures (Kesmen, Yetiman, Sahin, & Yetim, 2012), porcine and bovine in minced meat mixtures (Iwobi et al., 2015), and murine in pork, beef, mutton, chicken and duck meats (Fang & Zhang, 2016). Real-time PCR technique has been established as a robust technique for the detection and identification of species with high specificity and sensitivity by these and other similar researches. An excellent review by Salihah, Hossain, Lubis & Ahmed (2016) has summarized the use of real time PCR in food analysis.

Although method utilizing conventional PCR was proven to be successful, it requires a post-PCR manipulation that extends analysis time and handling hazardous chemical that may cause laboratory contamination. On the other hand, real-time PCR methods posses a great potential to replace the conventional PCR. This is mainly because real-time PCR methods are rapid, sensitive, specific, high degree of automation and target quantification (Heid et al, Genome Research, October 1996, Vol. 6, 986-994).

Real-time quantitative Polymerase Chain Reaction (PCR) is a DNA-based molecular technique that can amplify a species-specific region of the DNA with the aid of primers and probe. A hydrolysis probe has a reporter dye and one end and a quencher dye at the other end. During PCR process, the probe is hydrolysed and the reporter dye will fluoresce. The fluorescence emitted is recorded, indicating that the target DNA region is amplified. ZEN™ hydrolysis probe has a second internal quencher at about 9 base pairs away from the reporter dye. The double-quenchers in the ZEN™ probe reduce the background fluorescence to enhance the signal. Cal et al. (2012) also used the ZEN™ probe-based real-time PCR to detect porcine DNA in gelatine mixtures and in capsules. In contrast, present invention is a real-time qPCR assay based on the ZEN™ probe to detect and quantify porcine DNA in real processed meat samples.

Reference may be made from Patent Application CA2685133 which discloses pork-specific real-time PCR assay is developed for Halal authentication, however, it does disclose use of specific primers of present invention used in PCR. Also, it does not disclose use Zen probe which enhance the quantification of porcine DNA in real processed meat samples.

Another reference may be made from Pegels, Gonzalez, Fernandez, Garcia, & Martin (2012) titled “Sensitive detection of porcine DNA in processed animal proteins using a TaqMan real-time PCR assay” which relates to real-time PCR method was developed for specific detection of porcine-prohibited material in industrial feeds. The prior art does not disclose the specific primers and probe of present invention which enhance the quantification of porcine DNA in real processed meat samples.

Another reference may be made from Xia, Gravelsina, Ohrmalm, Ottoson, & Blomberg (2016) titled “ZEN™ Double-Quenched Probes add sensitivity and specificity to an assay for the highly variable noro virus” which disclose use of ZEN probe during qPCR. However, the prior art does not disclose detecting porcine DNA in food.

In terms of binary mixture, the lowest detected porcine adulteration was 0.01% by Ali et al. (2012) and is also achievable with Rapid Finder™ Pork ID Kit (Life Technologies). Based on previous studies, the detection times of 10 ng porcine DNA were 42.12 min (Rodriguez et al., 2005), 41.44 min (Kesmen et al., 2009), 13.75 min (Ali et al., 2012), 29.6 min (Cal et al., 2012), and 24.25 min (Yusop et al., 2012).

| | Method | | Sample | Detection | Limit of |
|---|---|---|---|---|---|
| | used | Target gene | type | time (min) | detection |
| Rodriguez et | TaqMan | 12S rRNA | Raw | 42.12 | 10 pg |
| al. (2005) | probe | | | | 0.5% (w/w) |
| Ali et al. | TaqMan | Cytochrome | Processed | 13.75 | 0.01% |
| (2012) | probe | b | | | (w/w) |
| Cammá et al. | TaqMan | Cytochrome | Raw | — | 0.8 pg |
| (2012) | probe | b | | | 1% (w/w) |
| Our method | ZEN | 12S rRNA | Raw and | 10.70 | 1 pg |
| | probe | | processed | | 0.001% |
| | | | | | (w/w) |

The present ZEN™ probe-based real-time qPCR assay is able to detect porcine DNA as low as 1 pg/μl in real food sample and as low as 0.001% porcine adulteration. The assay also rapidly detected 10 ng/μl of porcine DNA approximately in a much shorter time which was 10.70 minutes.

### SUMMARY OF THE INVENTION

The present invention is a process for identifying the porcine contamination from meat based raw and processed food sample wherein the process includes the steps of extracting deoxyribonucleic acid (DNA) from a food sample and amplifying a specific region of porcine (Sus scrofa) mitochondrial 12S ribosomal RNA gene by using forward primer having a DNA sequence of SEQ ID No. 1

5′GCCTAGCCCTAAACCCAAATAG3′

and a reverse primer having a DNA sequence of SEQ ID No. 2

5′GCAAGGGTTGGTAAGGTCTATC3′

where the specific porcine gene amplicon of 156 bp is identified by ZEN™ probe having a DNA sequence of SEQ ID No. 3

FAM/5′CTCTAGGTG/ZEN/GATGTGAAGCACCGC/3′IABk-FQ

In one aspect of the present invention provides a set of oligonucleotides primers of SEQ ID No. 1 and SEQ ID NO. 2.

In another aspect of the present invention provides a ZEN™ probe of SEQ ID NO. 3.

Yet another aspect of the present invention provides a kit containing at least a pair of forward and reverse primers of SEQ ID 1 and SEQ ID NO. 2 respectively, a probe of SEQ ID NO. 3 and instructions manual for using the kit in a specific manner to detect the porcine contamination from the meat based raw and processed food.

In yet another aspect of the present invention provides a method for manufacture the kit for detection of porcine contamination from the meat based raw and processed food.

### BRIEF DESCRIPTION OF THE DRAWINGS

FIG. 1 illustrates a flowchart of a method for rapidly identifying porcine contamination.

FIG. 2 shows the target fragment of porcine (Sus scrofa) mitochondrial 12SrRNA gene sequence (accession number AJ002189.1)

FIG. 3 shows the result and conclusion of specificity of the designed primers and probe

FIG. 4 the schematic of the chemistry of ZEN™ probe chemistry.

FIG. 5 shows the specificity of the designed primers and probe used in the present process. The Ct values of primers checked against ten folds serial DNA dilutions

FIG. 6 shown the sensitivity of the designed primers and probe. The Ct values of primers checked against ten folds serial DNA dilutions.

FIG. 7 shows amplification plot for the real processed food samples containing porcine.

FIG. 8 shows quantitative PCR analysis of complex mixtures from meat based products.

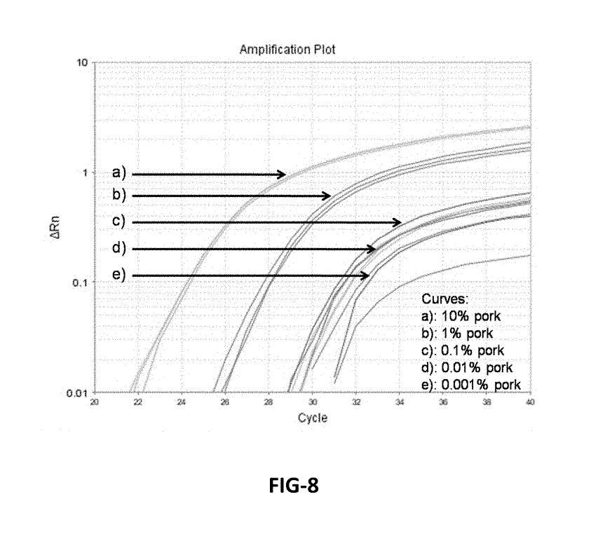

### DETAILED DESCRIPTION OF THE INVENTION

The invention describes the development and application of pork-specific real-time Polymerase Chain Reaction (PCR) assay for Halal authentication.

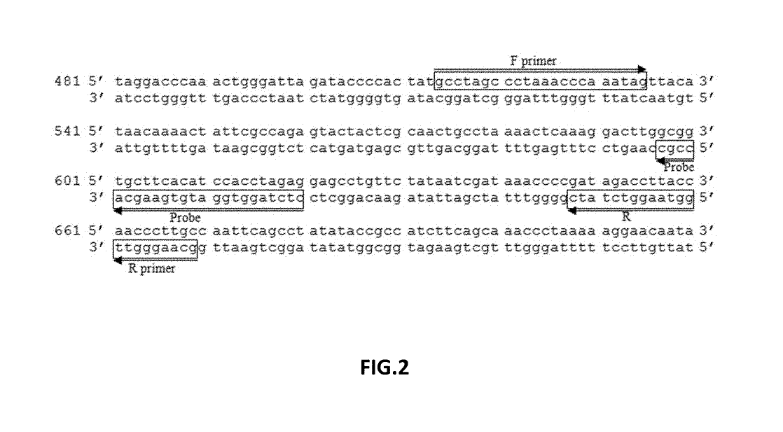

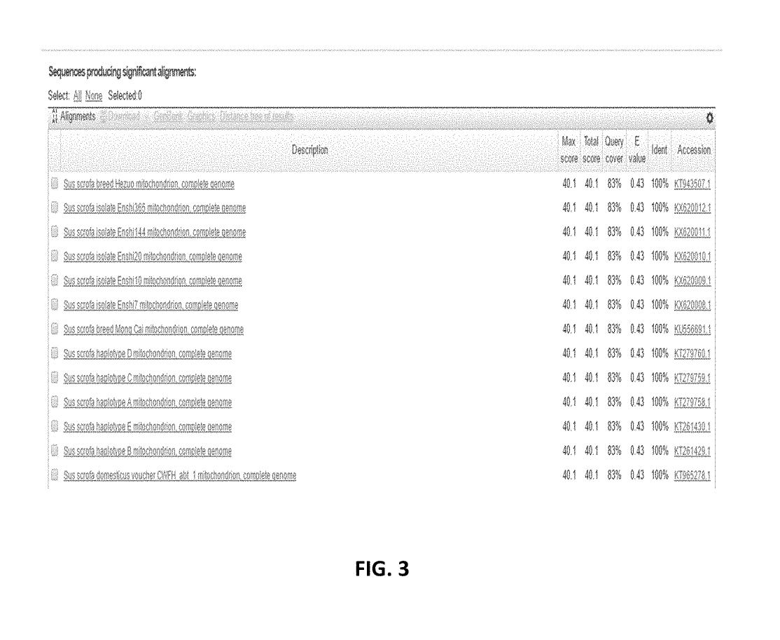

The invention may be best understood by reference to the following description, taken in conjunction with the accompanying figures. These figures and the associated description are provided to illustrate some embodiments of the invention, and not to limit the scope of the invention. In the following the invention will be described in greater detail with reference to exemplary embodiments in accordance with the accompanying drawings, in which-FIG. 3 shows the novel primers of SEQ ID NO. 1 and SEQ ID NO. 2 specific to the porcine mitochondrial DNA that were used for two reasons—firstly, mitochondrial DNAs shown in FIG. 2 are abundant in majority of the cells (Nakyinsige et al., 2012) and secondly, there are sufficient copies of mitochondrial DNA even when the genomic DNAs get degraded (Karabasanavar et al., 2014).

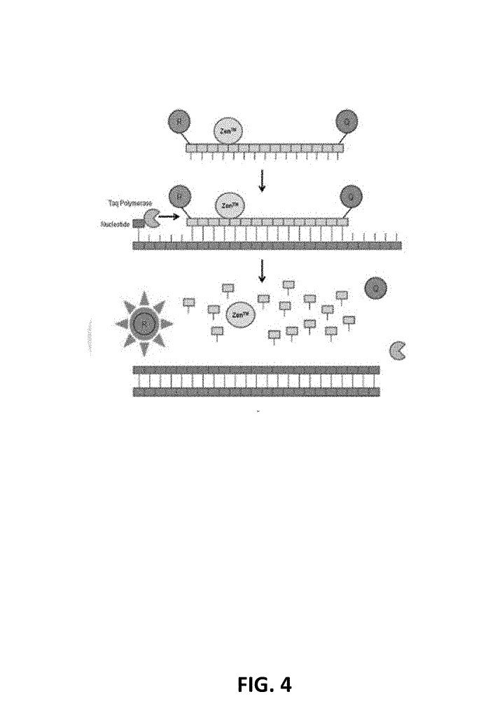

Further, for porcine detection the novel ZEN™ hydrolysis probe of SEQ ID NO. 3 was used with a reporter dye at one end and a quencher at the other end which shows almost similar functionality to the Taqman probe. Reference may be made from FIG. 4, the ZEN™ probe has a second internal ZEN quencher, at about 9 base pairs away from the reporter dye. The double-quenchers in the ZEN™ probe reduce the background fluorescence to enhance the signal (Integrated DNA technologies [IDT], 2011). The schematic of ZEN™ probe chemistry is illustrated in FIG. 4.

Cai et al. (2012) also used the ZEN™ probe-based real-time PCR to detect porcine DNA in gelatine mixtures and in capsules. In contrast, here, in the present invention, the development of a real-time qPCR assay based on the ZEN™ probe is shown to detect and quantify porcine DNA in real processed meat samples. In the present invention, Applicant has assessed the factors such as rapidity, specificity, sensitivity and applicability of the porcine which are related to detection and quantification of porcine contamination by real-time qPCR assay.

A process of the present invention may, if desired, be presented in a kit (e.g., a pack contain a materials for performing PCR) which may contain at least a set of oligonucleotide primers of SEQ ID NO. 1 & 2 and a probe of SEQ ID NO. 3 etc. The pack may for example comprise metal or plastic foil. The pack may be accompanied by instructions for performing detection and its use thereof.

The kit may also include at least one detection reagent that detects the presence of an isolated nucleic acid corresponding to porcine (Sus scrofa) mitochondrial 12S ribosomal RNA gene. For example, the kit may include probes or oligonucleotide sequences. The kit may contain in separate containers set of primers or a probe, control formulations (positive and/or negative), and/or a detectable label.

Instructions manuals (e.g., written, tape, VCR, CD-ROM, etc.) for carrying out the assay may be included in the kit. The assay may for example be in the form of real time-qPCR, as known in the art.

| | Our Method | Commercial Kit |
|---|---|---|
| Highly | Highly sensitive; detect as low | Sensitivity is 0.01% |
| sensitive | as 0.001% pork meat | pork meat |
| DNA | Porcine DNA can easily be | Requires additional kit |
| quantification | quantified using series of | (sold separately) |
| | standard DNA | |
| Shorter thermal | 22.3 min | 55 min |
| cycling time | | |
| Cost effective | Raw materials (reagents) are | Kit is expensive and can |
| | cheap and can be used for | only be used for 48 |
| | hundreds of reactions | reactions |

In one of its preferred embodiment of the present invention a method for manufacture the kit for use in detection of porcine contamination from food product sample is provided. The said method involves the steps of providing the means to isolate DNA of target fragment of the porcine (Sus scrofa) mitochondrial 12S ribosomal RNA gene, providing the set of primers of SEQ ID No. 1 and SEQ ID No. 2 which specifically bind to porcine (Sus scrofa) mitochondrial 12S ribosomal RNA gene; providing means to carry out amplification of the isolated DNA; providing probe of SEQ ID No.3 to determine the predetermined amplicon of 156 bp followed by evaluating a fluorescent signal in a real time PCR.

The following detailed description is merely exemplary in nature and is to enable any person skilled in the art to make and use the invention. The examples shown in description are not intended to limit the application and uses of the various embodiments. Various modifications to the disclosed invention will be readily apparent to those skilled in the art, and the methodology defined herein may be applied to other embodiments and applications without departing from the spirit and the scope of the present disclosure.

### Materials and Methods

### Materials

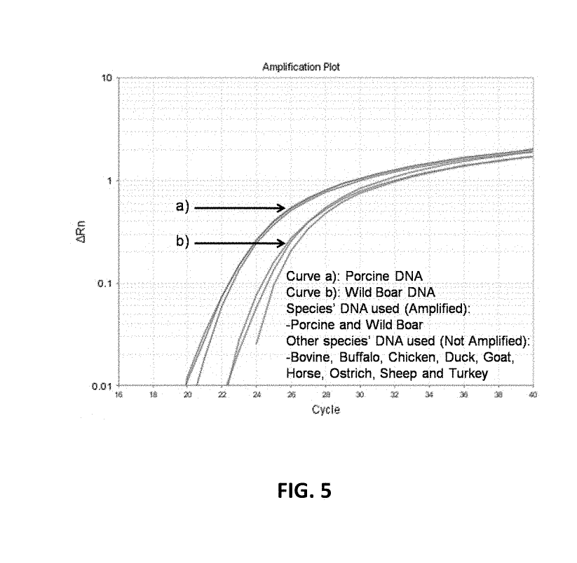

29 food samples produced in different countries were purchased from a local supermarket. In addition, raw pork and chicken meats were used to prepare 10%, 1%, 0.1%, 0.01% and 0.001% pork-in-chicken binary mixes for DNA extraction. DNA extracted from pork lean meat was used as positive porcine DNA control and ten-fold serial dilutions of the porcine DNA were prepared for determining the assay's sensitivity as illustrated in FIG. 5.

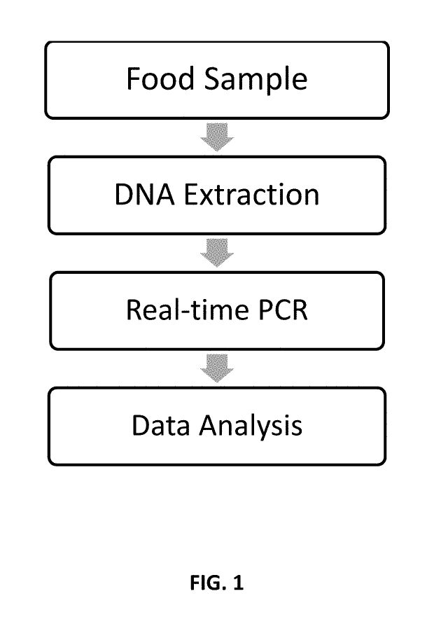

Following steps were followed in accordance with FIG. 1 for rapidly identifying porcine contamination.

### Example 1

### DNA Extraction

Immediately after opening the can or packaging of food samples, DNA was extracted from approximately 200 mg of each homogenized meat products and binary meat mixtures according to the user manual of the NucleoSpin® Food kit (Macherey-Nagel GmbH & Co. KG, Düren, Germany). Briefly, the homogenized sample was incubated with Proteinase K and lysis buffer at 65° C. for 30 minutes followed by centrifugation. The supernatant was mixed with binding buffer and ethanol prior to loading into a spin column. The column was washed three times with wash buffers before the DNA is eluted from the column using elution buffer. The concentration and purity of the extracted DNA were measured by Spectrophotometric method on Nano Photometer™ P-Class (Implen, Munchen, Germany). The DNA concentrationwas read at the absorbance of 260 nm while its purity was determined from the optical density of A260/A280 ratio. The extracted DNA was stored at −20° C. for use of real-time PCR assays.

### Example 2

### Primers and Probe Design

Reference may be made from the FIG. 3, where the species-specific primers were designed to target a fragment of the porcine (Sus scrofa) mitochondrial 12S rRNA gene of FIG. 2. The 12SrRNA gene sequence (accession number AJ002189.1) was retrieved from NCBI database (http://www.ncbi.nlm.nih.gov) and shown in FIG. 2. The specificity of the designed primers and probe were analyzed in silico using the BLAST tool in NCBI(http://blast.ncbi.nlm.nih.gov/Blast.cgi) to ensure that the sequences are specific to porcine species and to prevent non-specific binding to DNAs of other species as shown in FIG. 3.

Based on the analysis of result given in FIG. 3 the forward primer was designed as SEQ ID NO. 1 comprised of 22 nucleotides located between 444 and 466 nucleotides of porcine (Sus scrofa) mitochondrial 12S rRNA gene and the reverse primer was designed as SEQ ID NO. 2 comprised of 22 nucleotides located between 578 and 600 of porcine (Sus scrofa) mitochondrial 12S rRNA gene.

The ZEN™ probe is labeled with a fluorescent dye FAM and Iowa black FQ quencher at the 5′ end and 3′ end, respectively but not limited to it. An internal quencher ZEN is placed in the middle of the probe. The primers and probe were obtained from IDT (Integrated DNA Technologies, IDT PTE, Singapore).

The sequence of the oligonucleotide primers and probe are presented in Table 1.

TABLE 1
Oligonucleotide primers and probe

| | | | Amplicon |
|---|---|---|---|
| SEQ ID | Designation | Sequence (5′-3′) | size (bp) |
| SEQ ID | Forward | GCCTAGCCCTAAACCCAAATAG | 156 |
| NO. 1 | Primer | | |
| SEQ ID | Reverse | GCAAGGGTTGGTAAGGTCTATC | |
| NO. 2 | Primer | | |
| SEQ ID | Probe | FAM/CTCTAGGTG/ZEN/GATGTGAAGCACCGC/ | |
| NO. 3 | | 3IABkFQ/ | |

### Example 3

### Real-Time PCR Assay

The ZEN™ probe-based real-time qPCR amplifications were performed in 20 μl final reaction volumes which contained 1×PCR buffer II, 0.5 μM dNTP mix, 0.5 μM of each forward and reverse primer, 0.25 μM of ZEN™ probe, 0.1×ROX as passive reference, 0.5 U Ampli TaqDNA polymerase, 2 mM MgCl2 and 10 ng DNA.

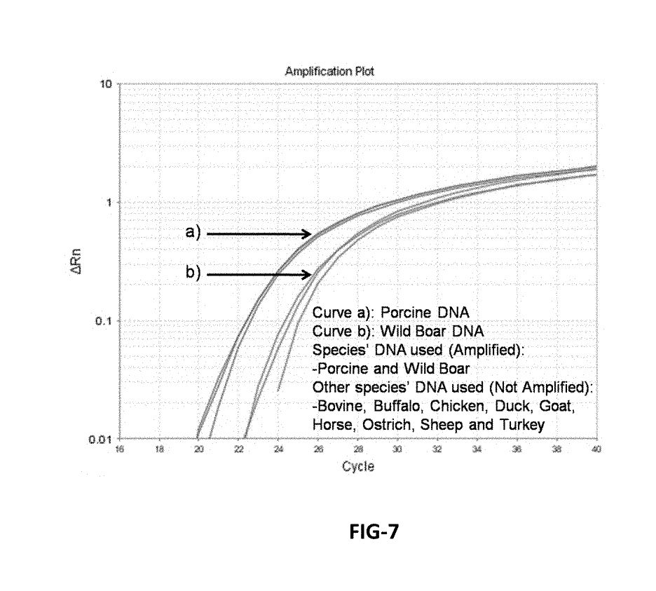

The real-time PCR reactions were performed on the 7500 Fast Real-Time PCR System (ABI); 7500 Software version 2.3 was used for data collection and processing. The fast thermal cycling profile used was 95° C. for 20 s followed by 40 cycles at 95° C. for 3 s and 60° C. for 30 s. FIG. 7 shows the amplification plot for the real processed food samples containing porcine.

### Example 4

### Specificity and Sensitivity Test

Reference may be made from FIG. 5, which shows the confirmation of specificity of the ZEN™ probe-based real-time qPCR assay using porcine specific primers of SEQ ID NO. 1 and SEQ ID NO. 2 by testing it with genomic DNAs from bovine, buffalo, chicken, duck, goat, horse, ostrich, sheep, turkey and wild boar, where the result of which can be analyzed through FIG. 5 and table 2.

TABLE 2
Results of the cross-reactivity test of the real-time qPCR
assay

| | | | Number of |
|---|---|---|---|
| | | | positive |
| Species DNA^{a} | Mean Ct value^{b} | SD^{c} | replicates |
| Porcine | 21.02 | 0.20 | 3/3 |
| Bovine | nd^{d} | Nd | 0/3 |
| Buffalo | nd | Nd | 0/3 |
| Chicken | nd | Nd | 0/3 |
| Duck | nd | Nd | 0/3 |
| Goat | nd | Nd | 0/3 |
| Horse | nd | Nd | 0/3 |
| Ostrich | nd | Nd | 0/3 |
| Sheep | nd | Nd | 0/3 |
| Turkey | nd | Nd | 0/3 |
| Wild Boar | 23.38 | 0.50 | 3/3 |

^{a}The DNA of different species were all made to have equal concentrations.
^{b}The Ct values are the mean of replicate assays (n = 3).
^{c}SD—standard deviation.
^{d}nd—porcine DNA not detected.

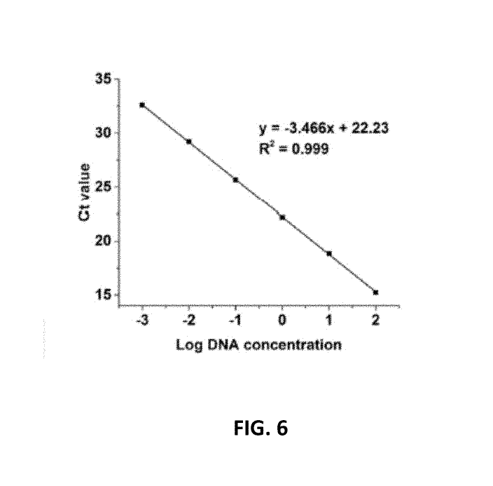

Sensitivity of the assay was measured by ten-fold serial dilutions of pork DNA extracted from pork lean meat ranging from 100 to 0.0001 ng/μl. The standard curve was generated by plotting the Ct values against the log of DNA concentrations as shown in FIG. 6 and table 3.

TABLE 3
Detection as low as 0.001% pork in raw-meat binary mixture

| | Mean Ct | | Positive | Concentration |
|---|---|---|---|---|
| Binary mixture (w/w) | value | SD | replicates | (ng/μl) |
| 0.001% pork in pork- | 30.86 | 1.5E−3 | 3/3 | 0.003 |
| chicken | | | | |
| 0.01% pork in pork- | 30.25 | 6.0E−4 | 3/3 | 0.004 |
| chicken | | | | |
| 0.1% pork in pork- | 30.45 | 2.1E−3 | 3/3 | 0.004 |
| chicken | | | | |
| 1% pork in pork- | 26.84 | 6.1E−3 | 3/3 | 0.044 |
| chicken | | | | |
| 10% pork in pork- | 23.11 | 1.1E−1 | 3/3 | 0.542 |
| chicken | | | | |

### Example 5

### Real Food Sample Analysis

The novel real-time PCR assay was used to detect the presence of pork DNA in 29 processed food samples which comprised of either pork or non-pork meat.

### Analysis of Extracted DNA

The concentrations of DNAs extracted from the food samples and from pork-chicken binary mixtures respectively ranged from 10 to 258 ng/μl and from 226 to 664 ng/μl. The purity of extracted DNA ranged from 1.42 to 1.95 for the food samples and from 1.96 to 2.02 for chicken mixtures. Despite some DNA yields were low, the overall DNA purity (A260/A280) was high which suggested the high quality of obtained DNA samples (Ali et al., 2015). It indicated the acceptability of the commercial DNA extraction kit for extracting DNA from canned meat products and raw meat mixtures.

TABLE 4
Results of the real-time PCR assay for the meat food samples

| Sample | | Mean | | Number of positive | Presence of |
|---|---|---|---|---|---|
| no. | Sample type^{a} | Ctvalue^{b} | SD^{c} | replicates | porcine DNA^{d} |
| 1 | Chopped pork and ham | 22.31 | 0.06 | 3/3 | + |
| 2 | Spiced pork cubes | 21.87 | 0.04 | 3/3 | + |
| 3 | Cocktail skinless | 20.89 | 0.00 | 3/3 | + |
| | sausages | | | | |
| 4 | Pork luncheon meat | 21.31 | 0.03 | 3/3 | + |
| 5 | Pork mince with | 21.64 | 0.01 | 3/3 | + |
| | bean paste | | | | |
| 6 | Pork luncheon meat | 24.12 | 0.06 | 3/3 | + |
| | with black pepper | | | | |
| 7 | Pork and bamboo | 26.69 | 0.20 | 3/3 | + |
| | shoot | | | | |
| 8 | Sliced ham | 22.89 | 0.01 | 3/3 | + |
| 9 | Pork short sausages | 19.08 | 0.04 | 3/3 | + |
| 10 | Marshmallow (Brand: | nd^{e} | nd | 0/3 | − |
| | Betta) | | | | |
| 11 | Marshmallow (Brand: | nd | nd | 0/3 | − |
| | Haribo) | | | | |
| 12 | Marshmallow (Brand: | nd | nd | 0/3 | − |
| | Mello Pastel) | | | | |
| 13 | Beef meat loaf | nd | nd | 0/3 | − |
| 14 | Corned beef (Brand: | nd | nd | 0/3 | − |
| | Sabli Food Industries) | | | | |
| 15 | Chicken frankfurters | nd | nd | 0/3 | − |
| 16 | Chicken luncheon | nd | nd | 0/3 | − |
| | Meat (Brand: Tulip) | | | | |
| 17 | Corned beef (Brand: | nd | nd | 0/3 | − |
| | Argentina) | | | | |
| 18 | Chicken luncheon | nd | nd | 0/3 | − |
| | meat (Brand: Mei | | | | |
| | Ning) | | | | |
| 19 | Mutton luncheon | nd | nd | 0/3 | − |
| | with chicken | | | | |
| 20 | Corned beef (Brand: | nd | nd | 0/3 | − |
| | Banquet) | | | | |
| 21 | Beef loaf | nd | nd | 0/3 | − |
| 22 | Corned mutton | nd | nd | 0/3 | − |
| 23 | Chicken luncheon | nd | nd | 0/3 | − |
| | Meat (Brand: Hana) | | | | |
| 24 | Beef curry | nd | nd | 0/3 | − |
| 25 | Chicken luncheon | nd | nd | 0/3 | − |
| | Meat (Brand: Golden | | | | |
| | Bridge) | | | | |
| 26 | Corned ostrich | nd | nd | 0/3 | − |
| 27 | Lamb curry with | nd | nd | 0/3 | − |
| | potatoes | | | | |
| 28 | Duck meat | nd | nd | 0/3 | − |
| 29 | Beef luncheon meat | nd | nd | 0/3 | − |

^{a}All samples were processed food products bought from local supermarkets manufactured indifferent countries,
^{b}The Ct values are the mean of replicate assays (n = 3).
^{c}SD—standard deviation.
dResult of real-time PCR: +, positive for porcine DNA; −, negative for porcine DNA.
^{e}nd—porcine DNA not detected.

### Specificity

In order to confirm the specificity of the present process to porcine species, DNA from ten other species were tested alongside porcine DNA as shown in Table 2.

Reference may be made from table 2 where no amplifications occurred for any of the ten species except for porcine and wild boar. Therefore, the process developed was very specific to porcine and wild boar species.

### Sensitivity

The sensitivity of the assay was determined by testing ten-fold serially diluted DNA templates starting with 10 ng/μl. The seven different DNA concentrations used were 10 ng/μl, 10 ng/μl, 1 ng/μl, 0.1 ng/μl, 0.01 ng/μl, 0.001 ng/μl, and 0.0001 ng/μl. Amplifications were observed for all DNA templates except for the lowest concentration of 0.0001 ng/μl. The standard curve from the amplifications of six DNA dilutions (FIG. 6) showed a very good linear regression with a perfect correlation coefficient (R2) of 0.999; the slope of the curve was −3.466. The assay showed an acceptable 94.3% efficiency for real time PCR (Yusop et al., 2012) which was calculated by using the formula E (%)=(10−1/slope−1)×100 (Cammá et al., 2012). The quantification range of this assay was therefore 100 ng/μl to 0.001 ng/μl and the lowest detectable DNA for this novel real-time qPCR assay was 0.001 ng/μl or 1 pg/μl. The detection limit attained in this study is comparable to that obtained in probe-based study (Cammá et al., 2012) and is lower than that obtained in a dye-based qPCR study (Soares et al., 2013) or in the “end-point” PCR study (Ali et al., 2015).

The standard curve was used to detect and quantify pork DNA from five pork-chicken raw meat binary mixtures. Raw meats were preferred as less time would be spent on sample preparation and as it was previously noted that cooking the meat did not give significant difference to raw meat in terms of Ct values (Kesmen et al., 2012). The mean Ct values of 11 pork-chicken binary mixtures ranged from 23.1 to 30.9 (Table 5).

TABLE 5
Results of the real-time PCR quantitative assay for the binary mixtures

| | | | Number of | |
|---|---|---|---|---|
| Binary mixture | Mean Ct | | positive | Concentration |
| (w/w)^{a} | value^{b} | SD^{c} | replicates | (ng/μl)^{d} |
| 0.001% pork in | 30.86 | 1.5E−3 | 3/3 | 0.003 |
| pork chicken | | | | |
| 0.01% pork in | 30.25 | 6.0E−4 | 3/3 | 0.004 |
| porkchicken | | | | |
| 0.1% pork in | 30.45 | 2.1E−3 | 3/3 | 0.004 |
| pork-chicken | | | | |
| 1% pork in | 26.84 | 6.1E−3 | 3/3 | 0.044 |
| pork-chicken | | | | |
| 10% pork in | 23.11 | 1.1E−1 | 3/3 | 0.542 |
| pork-chicken | | | | |

^{a}The binary mixtures of raw meat which we bought from local super markets.
^{b}The Ct values are the mean of replicate assays (n = 3).
^{c}SD—standard deviation.
^{d}The concentrations were determined based on standard curve.

It can be noted that the Ct value decreases as pork contaminant increases. Based on the result, the lowest percentage of pork contamination detected was 0.001%. This result is lower than that obtained in other studies (Rodriguez et al., 2005; Ali et al., 2012; Demirhan et al., 2012; Cammá et al., 2012; Cai et al., 2012; Yusop et al., 2012; Soares et al., 2013; Iwobi et al., 2015).

Based on the thermal cycling profile and the Ct value, the detection time for porcine DNA can be cross-calculated. The thermal cycling profile used was 20 s (at 95° C.) followed by 40 cycles of 3 s (at 95° C.) and 30 s (at 60° C.). In this process the mean Ct value for 10 ng porcine DNA was 18.85, meaning that the porcine DNA was detectable at cycle 18.85. By replacing the 40 cycles with 18.85 into the thermal profile, the time taken can be calculated as follows:

20 s+18.85 (3 s+30 s)=642.05 s (10.70 minute). Therefore, at 10.70 minute, 10 ng porcine DNA can already be detected. This detection time is comparatively faster than those from other studies (Rodriguez et al., 2005; Kesmen et al., 2009; Ali et al., 2012; Cal et al., 2012; Yusop et al., 2012).

A comparison of method used, target gene used, sample type, qPCR reaction time, detection time, limit of detection and limit of quantification of selected published studies, commercial real-time PCR kits, and this study are presented in Table 6. In brief, Applicant has developed new type of primers and probe for porcine detection in processed food samples and the present invention has several merits, for example, the detection time was quite low for 10 ng and 0.001 ng DNA was 10.70 and 18.27 min, and Also, the porcine contamination as low as 0.001% can e detected and quantified by applying present process as shown in table 6.

TABLE 6
Comparisons of selected published articles, commercial kit and
this study on porcine-related real-time qPCR

| | | | | Detection | |
|---|---|---|---|---|---|
| | Method | Target | Sample | time | Limit of |
| | used^{a} | gene^{b} | type^{c} | (min)^{d} | detection^{e} |
| Rodriguez | TaqMan | 12S | Raw | 42.12 | 10 pg, |
| et al. | probe | rRNA | | | 0.5% |
| (2005) | | | | | (w/w) |
| Kesmen et al. | TaqMan | ND5 | Raw and | 41.44 | 0.1 pg |
| (2009) | probe | | processed | | |
| Ali et al. | TaqMan | Cyto- | Processed | 13.75 | 0.01% |
| (2012) | probe | chrome | | | (w/w) |
| 0.01% (w/w) | | b | | | |
| Cai et al. | ZEN | Porcine | Processed | 29.6 | 0.1 pg, |
| (2012) | probe | repetitive | | | 1% |
| | | element | | | (w/w) |
| | | MPRE42 | | | |
| Cammá et al. | TaqMan | Cyto- | Raw | NA^{f} | 0.8 pg, |
| (2012) | probe | chrome | | | 1% |
| | | b | | | (w/w) |
| Yusop et al. | Molec- | Cyto- | Raw | 24.25 | 0.1 pg, |
| (2012) | ular | chrome | | | 0.1% |
| | beacon | b | | | (w/w) |
| | probe | | | | |
| Soares et al. | SYBR | Cyto- | Raw and | NA | 5 pg, |
| (2013) | Green | chrome | processed | | 0.1% |
| | dye | b | | | (w/w) |
| Iwobi et al. | TaqMan | Beta | Raw | NA | 50 pg, |
| (2015) | probe | actin | | | 1% |
| | | | | | (w/w) |
| Cycle | Chimeric | Cyto- | Processed | NA | NA |
| avePCR ™ | probe | chrome | | | |
| Meat Species | | oxidase | | | |
| Identification | | subunit I | | | |
| Kit | | | | | |
| (Takara Bio | | | | | |
| Inc.) | | | | | |
| Rapid | TaqMan | NA | Processed | NA | |
| Finder ™ | probe | | | | |
| Pork ID Kit | | | | | |
| (Life Tech- | | | | | |
| nologies) | | | | | |
| RealLine | TaqMan | NA | Processed | NA | NA |
| Food | probe | | | | 10 ng |
| Kit-Pork | | | | | |
| Detect | | | | | |
| (Bioron | | | | | |
| GmbH) | | | | | |
| Present | ZEN | 12S | Raw and | 10.70 | 1 pg, |
| invention | probe | rRNA | processed | | 0.001% |
| | | | | | (w/w) |

^{a}The type of probe used in the study.
^{b}Mitochondrial gene were targeted in these studies, unless otherwise stated in the table.
^{c}Sample type used: raw for raw meat, processed for either meat or other food type.
^{d}Time taken in minutes, based on the Ct value, to detect 10 ng porcine DNA
^{e}The limit of detection is for the raw or processed food and mixtures thereof.
^{f}NA—data not available or not stated.

### Applicability

In order to ensure that the developed novel ZEN™ probe-based real-time PCR assay is applicable, 29 commercially available food samples were successfully tested for the presence f porcine DNA. The assay detected porcine DNA in 10 out of the 29 food samples tested (Table 3). Based on the amplification curves of the DNA from pork meat products (FIG. 7), the Ct values of the pork meat products ranged from 19.1 to 26.7. This showed that the developed assay is able to detect and identify the presence of porcine DNA from processed meat samples despite concerns that processed food containing complex matrices of substances may inhibit PCR (Kesmen et al., 2009). The DNA extraction method where the binding of DNA to the silica membranes in the spin column in the presence of chaotropic agents was used may have removed the PCR inhibitors (Di Pinto, Forte, Conversano, & antillo, 2005).

1. A process for detection of porcine contamination from food product sample, wherein the said process comprising the following steps:
a) providing a product sample to be assayed;
b) isolating a DNA sample from the product sample specific to a biospecies of interest;
c) concentrating and purifying the isolated DNA sample corresponding to a 12S ribosomal RNA gene of step (b);
d) carrying out a polymerase chain reaction (PCR) on said DNA sample of step c) by using forward and reverse primer pair in order to amplify the DNA sequence, characterized in that the forward primer comprising a DNA sequence of SEQ ID No. 1
5′GCCTAGCCCTAAACCCAAATAG3′
and reverse primer comprising a DNA sequence of SEQ ID No. 2
5′GCAAGGGTTGGTAAGGTCTATC3′;
and
e) determining whether, or the extent to which, the predetermined amplicon is present in the amplified DNA product in order to detect the porcine contamination.

2. The process of claim 1, wherein the food product sample is a meat-based raw and processed food.

3. The process of claim 1, wherein the DNA sample is porcine DNA.

4. The process of claim 1, wherein the determination of presence of the predetermined amplicon in the amplified DNA product comprises:
adding a fluorogenic probe in the PCR amplification step d); and
evaluating a fluorescent signal for a PCR cycle at which it crosses a baseline.

5. The process of claim 1, wherein fluorogenic probe used is a ZEN™ probe with a reporter dye at 5′end and a quencher at 3′end.

6. The process of claim 5, wherein the ZEN™ probe comprises a DNA sequence of SEQ ID No. 3
FAM/5′CTCTAGGTG/ZEN/GATGTGAAGCACCGC/3′IABk-FQ
Wherein FAM stands for fluorescein and IABk stands for Iowa Black

7. The process of claim 5, wherein a reporter dye is selected from the group comprising FAM, HEX™, TET™, MAX and JOE etc.

8. The process of claim 5, wherein a quencher at 3′ end is selected from the group comprising ZEN-Iowa Black® FQ, Black Hole Quencher®, Eclipse®, Iowa Black FQ, and TAMRA

9. The process of claim 1, wherein the predetermined amplicon is no longer than 156 bp.

10. The process of claim 1, wherein the primer pairs used are species-specific primers to target a fragment of the porcine (Sus scrofa) mitochondrial 12S ribosomal RNA gene having an accession number AJ002189.1.

11. The process of claim 4, wherein the amplification is accomplished with quantitative real-time PCR.

12. The process of any of the claim 1, wherein the said process is able to identify the porcine contamination as low as 0.001%.

13. A set of oligonucleotide primers for the polymerase chain reaction amplification of DNA sequence corresponding to a target fragment of the porcine (Sus scrofa) mitochondrial 12S ribosomal RNA gene having an accession number AJ002189.1, wherein the said primers comprising:
forward primer having a DNA sequence of SEQ ID No. 1
5′GCCTAGCCCTAAACCCAAATAG3′;
and
reverse primer having a DNA sequence of SEQ ID No. 2
5′GCAAGGGTTGGTAAGGTCTATC3′

14. A set of oligonucleotide primers of claim 13, wherein the forward primer comprises at least 22 nucleotides positioned between 444 and 466 nucleotides and reverse primer comprises at least 23 nucleotides positioned between 578 and 600 nucleotides of target fragment of the porcine (Sus scrofa) mitochondrial 12S ribosomal RNA gene.

15. A novel probe comprising a DNA sequence of SEQ ID No. 3
FAM/5′CTCTAGGTG/ZEN/GATGTGAAGCACCGC/3′IABk-FQ
Wherein FAM stands for fluorescein and IABk stands for Iowa Black. FAM is the reporter dye and ZEN and IABk FQ are the quencher dyes. ZEN quencher is positioned after the ninth base from FAM reporter dye

16. The probe of claim 1, wherein the said probe is used to determine the presence of predetermined amplicon of 156 bp in the amplified DNA product corresponding to target fragment of the porcine (Sus scrofa) mitochondrial 12S ribosomal RNA gene.

17. (canceled)

18. (canceled)
